(19) United States
(12) Patent Application Publication
(10) Pub. No.: US 2025/0138001 A1
(43) Pub. Date: May 1, 2025

(54) Liposome-containing Diagnostic Reagents and Methods of Production and Use Thereof

(71) Applicant: Siemens Healthcare Diagnostics Inc., Tarrytown, NY (US)

(72) Inventors: Huey Lee, Millwood, NY (US); Frank Vitzthum, Biedenkopf (DE); Michelle Son, Hopewell Junction, NY (US); Luboslav Halik, White Plains, NY (US); William Bedzyk, Odessa, DE (US); Michael Sommer, Monroe, NY (US)

(73) Assignee: Siemens Healthcare Diagnostics Inc., Tarrytown, NY (US)

(21) Appl. No.: 18/837,283

(22) PCT Filed: Feb. 16, 2023
(86) PCT No.: PCT/US2023/062773

§ 371 Date: Aug. 9, 2024

Related U.S. Application Data
(60) Provisional application No. 63/317,643, filed on Mar. 8, 2022.

(51) Int. Cl. G01N 33/543; G01N 33/68

(52) U.S. Cl. CPC G01N 33/5432; G01N 33/68

(57) ABSTRACT

Diagnostic immunoassay reagent compositions are disclosed that include liposomes having analyte-specific binding element(s) associated therewith. In particular, the analyte-specific binding element(s) includes a transmembrane domain that anchors the element(s) in the lipid bilayer of the liposome; the analyte-specific binding element(s) further includes an extracellular/extra membrane domain that is exposed on the outer surface of the liposome and that comprises a domain that specifically binds to the target analyte. Also disclosed are kits, devices, and systems that contain the diagnostic immunoassay reagent compositions, as well as methods of producing and using the diagnostic immunoassay reagent compositions.

### CROSS REFERENCE TO RELATED APPLICATIONS/INCORPORATION BY REFERENCE STATEMENT

This application is a claims priority of U.S. Provisional application No. 63/317,643 filed 8 Mar. 2022, the contents of which are fully incorporated herein by reference.

### STATEMENT REGARDING FEDERALLY SPONSORED RESEARCH OR DEVELOPMENT

Not Applicable.

### BACKGROUND

In the fields of medicine and clinical chemistry, many studies and determinations of physiologically reactive species or analytes are carried out by taking advantage of the interaction between specific binding pair members. For example, the target analyte in a patient sample may be one member of a specific binding pair, and the target analyte is detectable by employing a corresponding member of the specific binding pair immobilized on a solid support. For example (but not by way of limitation), the immobilized binding pair member may be an antigen for the detection of a target antibody in a sample or vice versa (i.e., the immobilized binding pair member may be an antibody for detection of a target analyte in a sample), or the immobilized binding pair member may be a ligand for the detection of a target receptor in a sample or vice versa (i.e., the immobilized binding pair member may be a receptor for detection of a target ligand in a sample). Various support or surface materials have been developed for these applications and require various bonding or “functionalization” techniques to immobilize the specific binding pair member on the support/surface.

Surfaces functionalized with binders are commonly used as a basic test architecture for the detection of substances. Key problems encountered in the process of functionalizing surfaces with binders include the requirements of a large number of materials and resources as well as a substantial amount of time, in particular for “delicate” systems. Also, the coupling processes and waste material resulting therefrom may be hazardous, and the same is even true for certain surfaces that are applied. Management of respective processes and waste are typically associated with substantial safety measures as well as significant costs, too, and may even have to be discontinued for regulatory reasons with substantial consequences for a complete business.

For example, non-magnetic latex particles (NMLP) are widely used in homogeneous immunoassays as surfaces also referred to as solid phase. A binder, e.g., an antigen or antibody, is attached to these latex beads. The attachment can be performed by a chemical reaction. One non-limiting example of such attachment reaction involves the use of an azomethine reaction followed by a reduction process that can apply sodium cyanoborohydride (NaBH₃CN), a hazardous chemical compound that must be managed appropriately. The functionalized NMLPs and analytes of interest bind in a multivalent fashion, forming aggregates that can be visualized or quantified spectrophotometrically, in particular (but not by way of limitation) turbidimetrically or nephelometrically. Quantifying this change in the turbidity (or cloudiness) or scatter light of the reaction mixture as a function of an analyte concentration is the basis of a homogeneous immuno-agglutination assay.

Receptor proteins like thyroid stimulating hormone receptor (TSHR) on cellular membrane surfaces bind specific molecules or proteins to initiate a physiological change or response. TSHR is used for the detection of TSI (thyroid stimulating immunoglobulin) to aid in the diagnosis of Graves' disease. These receptors are generally transmembrane proteins and difficult to purify. The purification process tends to be elaborate and time-consuming. The purified receptor proteins are often unstable and need a relatively complex buffer system to maintain their function and stability. This makes the assay development difficult and hard to achieve.

Furthermore, attaching “delicate” binders, like thyroid stimulating hormone receptors to the latex beads for an immunoassay, normally requires a complex assay architecture with multiple components, for example (but not by way of limitation) the use of a secondary capture protein and a relatively complex buffer system to stabilize it and maintain its function.

In addition, these latex beads utilized in immunoassays eventually represent waste. The waste handling costs associated with these latex bead-containing immunoassay reagents will continue to be a challenge, and costs are likely to rise significantly in the future.

Therefore, there is a need in the art for new and improved diagnostic immunoassay reagents that overcome the defects and disadvantages of the prior art. It is to such reagents and kits and microfluidics devices containing same, as well as methods of producing and using same, that the present disclosure is directed.

### BRIEF DESCRIPTION OF THE DRAWINGS

FIG. 1 schematically depicts a diagram illustrating a portion of a diagnostic reagent composition constructed in accordance with the present disclosure. The diagnostic reagent composition comprises at least one receptor domain-containing binder on liposomes. A binder linked to the liposomes via a transmembrane domain can be a genetically engineered antigen, ligand, or antibody.

FIG. 2 schematically depicts a thyroid stimulating hormone receptor (TSHR) anchoring in the lipid bilayer of the liposomes via its transmembrane domain to form one non-limiting embodiment of a diagnostic reagent composition in accordance with the present disclosure. The thyroid stimulating lgG (TSI) epitope is present in the extracellular domain of the receptor protein.

FIG. 3 schematically depicts that TSHR-liposomes specifically recognized and bound TSI (anti-TSHR monoclonal antibody) in an agglutination-like event.

FIG. 4A contains dynamic light scattering profiles of liposomes functionalized with TSHR (TSHR-liposomes) in the absence and presence of an anti-TSHR monoclonal antibody (TSI). The observed size increase after adding the antibody demonstrates that an agglutination event was occurring.

FIG. 4B contains dynamic light scattering profiles of liposomes that are not functionalized with TSHR in the absence and presence of an anti-TSHR monoclonal antibody (TSI). No size increase is observed after adding the antibody to the liposomes. Thus, there is no agglutination. This demonstrates that the agglutination event shown in FIG. 4A is specific and only occurring, when functionalized TSHR-liposomes are used forming immune complexes with the TSI.

FIG. 5 schematically depicts other non-limiting embodiments of diagnostic reagent compositions constructed in accordance with the present disclosure. These embodiments include functionalized liposomes with and without dyes. The diagram illustrates the simultaneous detections of analyte A (square) and analyte B (circle) assisted by the presence or absence of the dyes in the liposomes.

### DETAILED DESCRIPTION

Before explaining at least one embodiment of the present disclosure in detail by way of exemplary language and results, it is to be understood that the present disclosure is not limited in its application to the details of construction and the arrangement of the components set forth in the following description. The present disclosure is capable of other embodiments or of being practiced or carried out in various ways. As such, the language used herein is intended to be given the broadest possible scope and meaning; and the embodiments are meant to be exemplary-not exhaustive. Also, it is to be understood that the phraseology and terminology employed herein is for the purpose of description and should not be regarded as limiting.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the articles, compositions, kits, and/or methods disclosed herein can be made and executed without undue experimentation in light of the present disclosure. While the articles, compositions, kits, and/or methods have been described in terms of particular embodiments, it will be apparent to those of skill in the art that variations may be applied to the articles, compositions, kits, and/or methods and in the steps or in the sequence of steps of the methods described herein without departing from the concept, spirit, and scope of the present disclosure. All such similar substitutes and modifications apparent to those skilled in the art are deemed to be within the spirit, scope, and concept of the present disclosure as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

The terms “analog” and “derivative” are used herein interchangeably and refer to a substance which comprises the same basic carbon skeleton and carbon functionality in its structure as a given compound, but can also contain one or more substitutions thereto. The term “substitution” as used herein will be understood to refer to the replacement of at least one substituent on a compound with a residue R. In certain non-limiting embodiments, R may include H, hydroxyl, thiol, a halogenid selected from fluoride, chloride, bromide, or iodide, a C1-C4 compound selected one of the following: linear, branched or cyclic alkyl, optionally substituted, and linear branched or cyclic alkenyl, wherein the optional substitutents are selected from one or more alkenylalkyl, alkynylalkyl, cycloalkyl, cycloalkenylalkyl, arylalkyl, heteroarylalkyl, heterocyclealkyl, optionally substituted heterocycloalkenylalkyl, arylcycloalkyl, and arylheterocycloalkyl, each of which is optionally substituted wherein the optional substitutents are selected from one or more of alkenylalkyl, alkynylalkyl, cycloalkyl, cyclalkenylalkyl, arylalkyl, alkylaryl, heteroarylalkyl, heterocyclealkyl, optionally substituted heterocycloalkenylalkyl, arylcycloalkyl, and arylheterocyclalkyl, phenyl, cyano, hydroxyl, alkyl, aryl, cycloalkyl, cyano, alkoxy, alkylthio, amino, —NH(alkyl), —NH(cycloalkyl)₂, carboxy, and —C(O))-alkyl.

The term “sample” as used herein will be understood to include any type of biological sample that may be utilized in accordance with the present disclosure. Examples of fluidic biological samples that may be utilized include, but are not limited to, whole blood or any portion thereof (i.e., plasma or serum), urine, saliva, sputum, cerebrospinal fluid (CSF), skin, intestinal fluid, intraperitoneal fluid, cystic fluid, sweat, interstitial fluid, extracellular fluid, tears, mucus, bladder wash, semen, fecal, pleural fluid, nasopharyngeal fluid, combinations thereof, and the like.

The term “specific binding partner,” as used in particular (but not by way of limitation) herein in the term “target analyte-specific binding partner,” will be understood to refer to any molecule capable of specifically associating with the target analyte. For example, but not by way of limitation, the binding partner may be an antibody, a receptor, a ligand, aptamers, molecular imprinted polymers (i.e., inorganic or organic matrices), combinations or derivatives thereof, as well as any other molecules capable of specific binding to the target analyte.

The term “antibody” is used herein in the broadest sense and refers to, for example, intact monoclonal antibodies and polyclonal antibodies, multi-specific antibodies (e.g., bispecific antibodies), antibody fragments and conjugates thereof that exhibit the desired biological activity of analyte binding (such as, but not limited to, Fab, Fab′, F(ab′)₂, Fv, scFv, Fd, diabodies, single-chain antibodies, and other antibody fragments and conjugates thereof that retain at least a portion of the variable region of an intact antibody), antibody substitute proteins or peptides (i.e., engineered binding proteins/peptides), and combinations or derivatives thereof. The antibody can be of any type or class (e.g., IgG, IgE, IgM, IgD, and IgA) or sub-class (e.g., IgG1, IgG2, IgG3, IgG4, IgA1, and IgA2).

An “analyte” is a macromolecule that is capable of being recognized by an analyte-specific binding partner, such as (but not limited to) an antibody. Both analytes and haptens comprise at least one antigenic determinant or “epitope,” which is the region of the antigen or hapten which binds to the analyte-specific binding partner (i.e., antibody). Typically, the epitope on a hapten is the entire molecule.

Turning now to the inventive concepts, certain non-limiting embodiments of the present disclosure are directed to a diagnostic reagent composition (such as, but not limited to, a diagnostic immunoassay reagent composition) for detection of a target analyte in a biological sample. The diagnostic reagent composition comprises a liposome and at least one analyte-specific binding element associated therewith. The liposome has a lipid bilayer and an outer surface, an inner surface, and a domain outside the membrane (i.e., an extramembrane domain). This extramembrane domain outside the membrane can reach into the space of the outer or inner surface of the liposome. The analyte-specific binding element comprises a transmembrane domain and an extracellular/extramembrane domain, and the extracellular/extramembrane domain comprises a domain that specifically binds to the target analyte. The liposome and analyte-specific binding element are associated such that the transmembrane domain of the at least one analyte-specific binding element is anchored in the lipid bilayer of the liposome; in this manner, at least a portion of the extramembrane domain of the analyte-specific binding element is exposed on the outer surface of the liposome for detection of the target analyte.

Any analyte-specific binding elements known in the art or otherwise contemplated herein that can be utilized for the diagnostic detection of binding of analyte thereto can be utilized in accordance with the present disclosure. Non-limiting examples of types of analyte-specific binding elements utilized for diagnostic use include receptors, ligands, antigens, antibodies, aptamers, molecularly imprinted polymers, and the like, as well as derivatives and variants thereof, and any combinations thereof.

The analyte-specific binding elements utilized in accordance with the present disclosure may be integrally formed with the transmembrane and extracellular/extramembrane domains attached to one another. Non-limiting examples of transmembrane-domain-containing proteins or portions thereof that may be utilized as analyte-specific binding elements in accordance with the present disclosure include receptor proteins or portions thereof. Particular non-limiting examples of receptor proteins and derivatives or variants thereof that may be utilized in accordance with the present disclosure to detect a target analyte present in a biological sample include at least a portion of thyroid-stimulating hormone receptor (TSHR), a ligand-gated ion channel receptor, an enzyme-linked or kinase-linked receptor, a cell-matrix adhesion receptor, a cell adhesion molecule, an adrenergic receptor, an IgG Fc receptor, a glucocorticoid receptor, a G protein-coupled receptor, and the like, as well as any combinations thereof.

Alternatively, the analyte-specific binding element may be created by covalently conjugating a homologous or heterologous transmembrane domain to a soluble protein/peptide or an extracellular fragment of a protein/peptide. Non-limiting examples of soluble proteins/peptides (including extracellular fragments thereof) that can be conjugated to a transmembrane domain include antigens, ligands, antibodies, and the like. Particular non-limiting examples of soluble protein/peptides that can be conjugated to a transmembrane domain to form the analyte-specific element for the detection of a target analyte present in a biological sample include prostate-specific antigen, an antibody against alpha-fetoprotein, an intrinsic factor, and the like, as well as any combinations thereof.

Any heterologous transmembrane domains known in the art or otherwise contemplated herein that is capable of being conjugated to soluble or extracellular protein/peptide can be utilized in accordance with the present disclosure. Non-limiting examples of transmembrane domains known in the art that are utilized to anchor soluble or extracellular proteins/peptides to lipids include the transmembrane domains of various ion channel receptors or other types of receptors (such as, but not limited to, G protein-coupled receptors (GPCRs)), transporters, and the like. In addition, the transmembrane domain can also be a part of a pore-forming protein in general. Non-limiting examples for pore-forming proteins that possess transmembrane domains that can be utilized in accordance with the present disclosure include porin, hemolysin, nucleoporin, membrane attack complex (MAC), complement component C9, or subunits, combinations, or multimers of porin, hemolysin, nucleoporin, membrane attack complex (MAC), and complement component C9, and the like.

The liposomes of the diagnostic reagent compositions of the present disclosure may be formed of any lipid(s) known in the art or otherwise contemplated herein and may possess any size, so long as the diagnostic reagent composition is capable of binding to analyte present in a biological sample and producing a detectable signal indicative of the presence and/or concentration of the target analyte within the biological sample. Upon binding to analyte, the diagnostic reagent composition may be capable of producing the detectable signal by itself (i.e., homogeneously) or via interaction with one or more other diagnostic reagents (i.e., heterogeneously).

Suitable liposomes for use in the compositions described in this application include (but are not limited to) multilamellar vesicles (MLVs), small unilamellar liposome vesicles (SUVs), large unilamellar liposome vesicles (LUVs), giant unilamellar liposome vesicles (GUVs), and the like.

Non-limiting examples of liposome sizes that may be utilized in accordance with the present disclosure include about 20 nm, about 25 nm, about 30 nm, about 35 nm, about 40 nm, about 45 nm, about 50 nm, about 60 nm, about 70 nm, about 75 nm, about 80 nm, about 90 nm, about 100 nm, about 110 nm, about 120 nm, about 125 nm, about 130 nm, about 140 nm, about 150 nm, about 160 nm, about 170 nm, about 175 nm, about 180 nm, about 190 nm, about 200 nm, about 210 nm, about 220 nm, about 225 nm, about 230 nm, about 240 nm, about 250 nm, about 260 nm, about 270 nm, about 275 nm, about 280 nm, about 290 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 675 nm, about 700 nm, about 725 nm, about 750 nm, about 775 nm, about 800 nm, about 825 nm, about 850 nm, about 875 nm, about 900 nm, about 925 nm, about 950 nm, about 975 nm, about 1000 nm, about 1225 nm, about 1250 nm, about 1275 nm, about 1300 nm, about 1325 nm, about 1350 nm, about 1375 nm, about 1400 nm, about 1425 nm, about 1450 nm, about 1475 nm, about 1500 nm, about 1600 nm, about 1700 nm, about 1800 nm, about 1900 nm, about 2000 nm, about 2100 nm, about 2200 nm, about 2300 nm, about 2400 nm, about 2500 nm, about 2600 nm, about 2700 nm, about 2800 nm, about 2900 nm, about 3000 nm, about 3100 nm, about 3200 nm, about 3300 nm, about 3400 nm, about 3500 nm, about 3600 nm, about 3700 nm, about 3800 nm, about 3900 nm, about 4000 nm, about 4100 nm, about 4200 nm, about 4300 nm, about 4400 nm, about 4500 nm, about 4600 nm, about 4700 nm, about 4800 nm, about 4900 nm, about 500 nm, and larger, as well as a range formed of any of the above values (i.e., a range of from about 20 nm to about 5000 nm, a range of from about 20 nm to about 1000 nm, a range of from about 50 nm to about 1000 nm, a range of from about 50 nm to about 500 nm, a range of from about 100 nm to about 500 nm, a range of from about 100 nm to about 200 nm, etc.).

Non-limiting examples of lipids that may be utilized to produce the liposomes include phospholipids such as (but not limited to), a phosphatidylcholine, a phosphatidylserine, a phosphatidylethanolamine, a phosphatidylglycerol, and the like, as well as derivatives thereof, and any combinations thereof.

Other non-limiting examples of types of lipids that can be utilized to produce the liposomes include sphingolipids, glycerophospholipids, sterols, and sterol derivatives. Non-limiting examples of sphingolipids that can be utilized include sphingomyelin and ceramides containing saturated, monounsaturated, and/or polyunsaturated acyl chains of different lengths. Non-limiting examples of phospholipids with various headgroup structures that can be utilized include phosphatidic acid (PA), phosphatidylcholine (PC), phosphatidylethanolamine (PE), phosphatidylglycerol (PG), phosphatidylinositol (PI), cardiolipin, phosphatidylserine (PS) containing saturated, monounsaturated, and/or polyunsaturated acyl chains of different lengths. Non-limiting examples of sterols and sterol derivatives that can be used include but not limited to cholesterol, brassicasterol, allocholesterol, cholesterol methyl ether, campestanol, campesterol, cholesteryl acetate, coprostanol, desmosterol, dehydrodesmosterol, dihydrocholesterol, dihydrolanosterol, epicholesterol, lathosterol, lanosterol, sitostanol, sitosterol, stigmasterol, zymostenol, zymosterol, and the like.

Mixtures of lipids can also be used, including mixtures of two or more of sphingolipids, glycerophospholipids, sterols, and sterol derivatives. In certain particular (but non-limiting) embodiments, sterols and sterol derivatives are not used alone to produce the liposomes, but rather are included in mixtures with sphingolipids or glycerophospholipids. In a particular (but non-limiting) embodiment, the sterols and/or sterol derivatives are present in sphingolipid-or glycerophospholipid-containing liposomes in a range of from about 0% to about 40% of total lipids.

Particular (but non-limiting) examples of sphingolipids that can be utilized in accordance with the present disclosure include porcine brain sphingomyelin, chicken egg sphingomyelin, and bovine milk sphingomyelin. Particular (but non-limiting) examples of glycerophospholipids that can be utilized in accordance with the present disclosure include phospholipids with various headgroup structures such as phosphatidylcholine (PC), phosphatidylethanolamine (PE), phosphatidylglycerol (PG), phosphatidylinositol (PI), cardiolipin, phosphatidylserine (PS) with two saturated acyl chains of different lengths (e.g., 1,2-dipalmitoyl-sn-glycero-3-phosphocholine, 1,2-distearoyl-sn-glycero-3-phosphocholine, 1,2-dipalmitoyl-sn-glycero-3-phosphoethanolamine, 1,2-distearoyl-sn-glycero-3-phosphoethanolamine, 1,2-dipalmitoyl-sn-glycero-3-phospho-(1′-rac-glycerol), 1,2-distearoyl-sn-glycero-3-phospho-(1′-rac-glycerol), 1,2-dipalmitoyl-sn-glycero-3-phospho-(1′-myo-inositol), 1,2-distearoyl-sn-glycero-3-phosphoinositol, 1′,3′-bis[1,2-dipalmitoyl-sn-glycero-3-phospho]-glycerol, 1′,3′-bis [1,2-distearoyl-sn-glycero-3-phospho]-glycerol, 1,2-dipalmitoyl-sn-glycero-3-phospho-L-serine, 1,2-distearoyl-sn-glycero-3-phospho-L-serine), and with one saturated acyl chain of different lengths and one monounsaturated acyl chain of different lengths (e.g.,1-palmitoyl-2-oleoyl-sn-glycero-3-phosphocholine, 1-stearoyl-2-oleoyl-sn-glycero-3-phosphocholine,1-palmitoyl-2-oleoyl-sn-glycero-3- phosphoethanolamine, 1-stearoyl-2-oleoyl-sn-glycero-3-phosphoethanolamine, 1-palmitoyl-2-oleoyl-sn-glycero-3-phospho-(1′-rac-glycerol), 1-stearoyl-2-oleoyl-sn-glycero-3-phospho-(1′-rac-glycerol), 1-palmitoyl-2-oleoyl-sn-glycero-3-phosphoinositol, 1′,3′-bis [1-palmitoyl-2-oleoyl-sn-glycero-3-phospho]-glycerol, 1-palmitoyl-2-oleoyl-sn-glycero-3-phospho-L-serine, 1-stearoyl-2-oleoyl-sn-glycero-3-phospho-L-serine), and with one saturated acyl chain of different lengths and one polyunsaturated acyl chain of different lengths (e.g., 1-palmitoyl-2-linoleoyl-sn-glycero-3-phosphocholine, 1-stearoyl-2-linoleoyl-sn-glycero-3-phosphocholine, 1-palmitoyl-2-linoleoyl-sn-glycero-3-phosphoethanolamine, 1-stearoyl-2-linoleoyl-sn-glycero-3-phosphoethanolamine, 1-palmitoyl-2-linoleoyl-sn-glycero-3-phospho-(1′-rac-glycerol), 1-stearoyl-2-linoleoyl-sn-glycero-3-phospho-(1′-rac-glycerol), 1-palmitoyl-2-linoleoyl-sn-glycero-3-phospho-L-serine, 1-stearoyl-2-linoleoyl-sn-glycero-3-phospho-L-serine). In certain particular (but non-limiting) examples, each fatty acid acyl chain has a number of carbon atoms ranging from about 16 to about 20, and particularly (but not by way of limitation) 16, 18, or 20 carbon atoms. A particular (but non-limiting) number of double bonds per each fatty acid acyl chain ranges from 0 to 2. Particular (but non-limiting) examples of sterols and sterol derivatives for use with sphingolipid-or glycerophospholipid-containing liposomes includes cholesterol, dihydrocholesterol, epicholesterol, sitosterol, and lathosterol. Cholesterol is the most particular (but non-limiting) sterol. The particular (but non-limiting) sphingolipids and glycerophospholipids can be used either alone or as a mixture of sphingolipids and glycerophospholipids in the presence of particular (but non-limiting) sterols and sterol derivatives to form the porous liposomes described in the present disclosure. For example, in particular (but non-limiting) embodiments, the liposomes can comprise 100% porcine brain sphingomyelin or a mixture of porcine brain sphingomyelin and cholesterol. In a particular (but non-limiting) example, sterols and sterol derivatives can be present in the range of from about 20% to about 40% of total lipids, such as (but not limited to) about 30%.

The diagnostic reagent composition may be provided with any element(s) or feature(s) that allow for detection of complexes of liposome with analyte bound thereto. In certain particular (but non-limiting) embodiments, the diagnostic reagent composition may have at least one dye associated therewith to facilitate detection of bound analyte. For example (but not by way of limitation), the liposome may have at least one dye encapsulated therein. Alternatively (and/or in addition thereto), at least one dye-containing lipid (such as, but not limited to, a dye-containing phospholipid) may be utilized in the production of the liposome or otherwise incorporated therein. Dyes could be used (for example, but not by way of limitation) for spectrophotometric, luminescence detection (i.e., chemiluminescence or fluorescence detection).

In particular (but non-limiting) embodiments, at least one biotinylated lipid (such as, but not limited to, a biotinylated phospholipid) may be utilized in the production of the liposome.

Certain non-limiting embodiments of the present disclosure are directed to kits that contain one or more of any of the diagnostic reagent compositions disclosed or otherwise contemplated herein. In certain particular (but non-limiting) embodiments, the kit further includes at least one additional assay reagent that interacts with the diagnostic reagent composition for detecting the presence and/or concentration of the target analyte in the biological sample.

In a particular (but non-limiting) embodiment, the kit comprises two or more diagnostic reagent compositions disposed together in a single composition for performing a multiplex assay for two different target analytes. In this manner, the first and second diagnostic reagent compositions are provided with different dyes associated therewith (or other types of detection mechanisms that differ from one another) that allow for detection of both target analytes in a single reaction.

The compositions/reagents of the kits may be provided in any form that allows them to function in accordance with the present disclosure. For example, but not by way of limitation, each of the reagents may be provided in liquid form and disposed in bulk and/or single aliquot form within the kit. Alternatively, in a particular (but non-limiting) embodiment, one or more of the reagents may be disposed in the kit in the form of a single aliquot lyophilized reagent. The use of dried reagents in kits/microfluidics devices is described in detail in U.S. Pat. No. 9,244,085 (Samproni), the entire contents of which are hereby expressly incorporated herein by reference.

In addition to the compositions/reagents described in detail herein above, the kits may further contain other reagent(s) for conducting any of the particular assays described or otherwise contemplated herein. The nature of these additional reagent(s) will depend upon the particular assay format, and identification thereof is well within the skill of one of ordinary skill in the art; therefore, no further description thereof is deemed necessary. Also, the compositions/reagents present in the kits may each be in separate containers/compartments, or various compositions/reagents can be combined in one or more containers/compartments, depending on the cross-reactivity and stability of the compositions/reagents. In addition, the kit may include a microfluidics device in which the compositions/reagents are disposed.

The relative amounts of the various compositions/reagents in the kits can vary widely to provide for concentrations of the compositions/reagents that substantially optimize the reactions that need to occur during the assay methods and further to optimize substantially the sensitivity and selectivity of an assay. Under appropriate circumstances, one or more of the compositions/reagents in the kit can be provided as a dry powder, such as a lyophilized powder, and the kit may further include excipient(s) for dissolution of the dried reagents; in this manner, a reagent solution having the appropriate concentrations for performing a method or assay in accordance with the present disclosure can be obtained from these compositions. Positive and/or negative controls may also be included with the kit. In addition, the kit can further include a set of written instructions explaining how to use the kit. A kit of this nature can be used in any of the methods described or otherwise contemplated herein.

Certain additional non-limiting embodiments of the present disclosure are directed to a microfluidics device that includes one or more of any of the diagnostic reagent compositions described herein above or otherwise contemplated herein. In particular, certain non-limiting embodiments include a microfluidics device for determining the concentration of at least one target analyte in a sample. The microfluidics device comprises: (i) an inlet channel through which a sample is applied; and (ii) at least a first compartment capable of being in fluidic communication with the inlet channel and containing at least one of the diagnostic reagent compositions disclosed or otherwise contemplated herein. The compartment(s) of (ii) may further contain any additional reagents required to perform the assay for detection of the target analyte. Any of the assay reagents disclosed or otherwise contemplated herein may be utilized in the microfluidics devices of the present disclosure.

The microfluidics device may be provided with any arrangement of compartments and distribution of the various compositions/reagents therebetween that allows the device to function in accordance with the present disclosure. That is, when the diagnostic reagent composition is utilized in combination with a second assay reagent, the two reagents may be disposed in the same compartment or in different compartments. When the two reagents are separated between two compartments, the diagnostic reagent composition may be disposed in a first compartment that is in fluidic communication with the inlet channel, and the at least one additional assay reagent may be disposed in a second compartment that is in fluidic communication with the first compartment.

In a particular (but non-limiting) embodiment, the microfluidics device comprises two or more of any of the diagnostic reagent compositions disposed together in the microfluidics device such that a multiplex assay for two different target analytes can be performed within the microfluidics device. In this manner, the first and second diagnostic reagent compositions (and additional diagnostic reagent compositions, if present) are provided with different dyes associated therewith (or other types of detection mechanisms that differ from one another) that allow for detection of both target analytes in a single reaction. The two or more diagnostic reagent compositions may be disposed in the same or separate compartments of the microfluidics device.

The microfluidics devices of the present disclosure may possess any design or configuration known in the art or otherwise contemplated herein for use in a diagnostic analyte assay (such as, but not limited to, a diagnostic immunoassay). In certain particular (but non-limiting) embodiments, the microfluidics device may be in the form of a cassette that is configured for insertion into an automated diagnostic test instrument system that performs the diagnostic assay. Alternatively, the microfluidics device may be a standalone product that can be read without a diagnostic test instrument system. For example (but not by way of limitation), the microfluidics device may be in the form of a lateral flow device that can be conducted at a point of care (POC) location.

Any of the compartments of the microfluidics device may be sealed to maintain reagent(s) disposed therein in a substantially air tight environment until use thereof; for example, compartments containing lyophilized reagent(s) may be sealed to prevent any unintentional reconstitution of the reagent. The inlet channel and a compartment, as well as two compartments, may be described as being “capable of being in fluidic communication” with one another; this phrase indicates that each of the compartment(s) may still be sealed, but that the two compartments are capable of having fluid flow therebetween upon puncture of a seal formed therein or therebetween.

The microfluidics devices of the present disclosure may be provided with any other desired features known in the art or otherwise contemplated herein. For example, but not by way of limitation, the microfluidics devices of the present disclosure may further include a read chamber; the read chamber may be any of the compartments containing the reagent(s) described herein above, or the read chamber may be in fluidic communication with said compartment. The microfluidics device may further include one or more additional compartments containing other solutions, such as (but not limited to) wash solutions, dilution solutions, excipients, interference solutions, positive controls, negative controls, quality controls, and the like. These additional compartment(s) may be in fluidic communication with one or more of the other compartments. For example, the microfluidics device may further include one or more compartments containing a wash solution, and these compartment(s) may be capable of being in fluidic communication with any other compartment(s) of the device. In another example, the microfluidics device may further include one or more compartments containing an excipient for dissolution of one or more dried reagents, and the compartment(s) may be capable of being in fluidic communication with any other compartment(s) of the device. In yet a further example, the microfluidics device may include one or more compartments containing a dilution solution, and the compartment(s) may be capable of being in fluidic communication with any other compartment(s) of the device.

Certain non-limiting embodiments of the present disclosure are directed to a method of producing any of the diagnostic reagent compositions disclosed or otherwise contemplated herein. In a particular (but non-limiting) embodiment, the method includes the following steps: (1) combining any of the lipids disclosed or otherwise contemplated herein with at least one of any of the analyte-specific binding elements disclosed or otherwise contemplated herein to form a mixture; and (2) incubating the mixture under conditions that result in the formation of at least one liposome having the at least one analyte-specific binding element anchored in a lipid bilayer of the liposome via the transmembrane domain of the at least one analyte-specific binding element, and wherein the extracellular/extramembrane domain of the at least one analyte-specific binding element comprises a domain that extends from an outer surface of the liposome for specifically binding to the target analyte.

Alternatively, the liposome may be formed prior to interaction with the analyte-specific binding element for producing the diagnostic reagent composition. Therefore, in another particular (but non-limiting) embodiment, the method comprises the steps of: (1) combining at least one of any of the liposomes disclosed or otherwise contemplated herein with at least one of any of the analyte-specific binding elements disclosed or otherwise contemplated herein to form a mixture; and (2) incubating the mixture under conditions that result in the anchoring of the at least one analyte-specific binding element in the lipid bilayer of the at least one liposome via the transmembrane domain of the at least one analyte-specific binding element, and wherein the extracellular/extramembrane domain of the at least one analyte-specific binding element comprises a domain that extends from the outer surface of the liposome for specifically binding to the target analyte.

In addition, in certain non-limiting embodiments, at least one dye may be incorporated into the diagnostic reagent composition during the method of production. For example (but not by way of limitation), when lipids are mixed with the analyte-specific binding element to form the liposomes, then at least one dye can be added to the mixture such that the at least one liposome formed has at least one dye encapsulated therein. Optionally, at least one dye-containing phospholipid and/or at least one biotinylated phospholipid may be utilized as one of the lipids that is mixed with the analyte-specific binding element to form the liposomes. In an alternative, when the analyte-specific binding element is mixed with preformed liposome, then the at least one liposome may have at least one dye encapsulated therein, or the at least one liposome may comprise at least one dye-containing phospholipid and/or at least one biotinylated phospholipid.

Certain non-limiting embodiments of the present disclosure are directed to a method of determining the presence and/or concentration of at least one target analyte in a biological sample. In the method, the biological sample is combined with at least one of any of the diagnostic reagent compositions disclosed or otherwise contemplated herein under conditions that allow target analyte present in the biological sample to substantially bind to the analyte-specific binding element extending from the outer surface of the liposome of the diagnostic reagent composition, thereby forming a complex; then, the presence and/or concentration of the target analyte is determined based on any complex formed by any assay methods known in the art.

In certain particular (but non-limiting) embodiments, the method utilizes a homogeneous assay format. For example (but not by way of limitation), binding of target analyte to the diagnostic reagent composition results in agglutination, and therefore no additional reagents are required for detection of target analyte.

Alternatively, the method may utilize a heterogeneous assay format, in which an additional reagent must be used in combination with the diagnostic reagent composition for detection of the target analyte in the biological sample. For example (but not by way of limitation), the method may utilize a heterogenous format such as a sandwich assay. When a second reagent must be utilized, the biological sample may be contacted with the diagnostic reagent composition and the second reagent either simultaneously or wholly or partially sequentially. In addition, the detection step of the method will involve detection of the complex comprising diagnostic reagent composition/target analyte/second assay reagent.

Certain non-limiting embodiments of the present disclosure are directed to a method of determining the presence and/or concentration of at least two target analytes in a biological sample. In the method, the biological sample is combined with at least two of any of the diagnostic reagent compositions disclosed or otherwise contemplated herein (i.e., a first diagnostic reagent composition and a second diagnostic reagent composition) under conditions that: (1) allow a first target analyte present in the biological sample to substantially bind to the analyte-specific binding element extending from the outer surface of the liposome of the first diagnostic reagent composition, thereby forming a first complex, and (2) allow a second target analyte present in the biological sample to substantially bind to the analyte-specific binding element extending from the outer surface of the liposome of the second diagnostic reagent composition, thereby forming a second complex. Then, the presence and/or concentration of each of the first and second target analytes is determined based on any first and second complexes, respectively, formed by any assay methods known in the art.

Non-limiting examples of biological samples that may be utilized in accordance with the various methods of the present disclosure include whole blood or any portion thereof (i.e., plasma or serum), urine, saliva, sputum, cerebrospinal fluid (CSF), skin, intestinal fluid, intraperitoneal fluid, cystic fluid, sweat, interstitial fluid, extracellular fluid, tears, mucus, bladder wash, semen, fecal, pleural fluid, nasopharyngeal fluid, and combinations thereof. Particular non-limiting examples include lysed whole blood cells and lysed red blood cells.

As mentioned above, when the diagnostic reagent composition is utilized in combination with a second reagent, the two compositions may be added either simultaneously or sequentially. In addition, when two or more diagnostic reagent compositions are utilized in the same reaction, the two diagnostic reagent compositions may be added simultaneously or sequentially. When the various compositions utilized in the method are added sequentially, the order of addition of the compositions may be varied; a person having ordinary skill in the art can determine the particular desired order of addition of the different compositions to the assay. The simplest order of addition, of course, is to add all the materials simultaneously and determine the signals produced therefrom. Alternatively, each of the compositions, or groups of compositions, can be combined sequentially. In certain embodiments, an incubation step may be involved subsequent to one or more additions.

### EXAMPLE

An Example is provided hereinbelow. However, the present disclosure is to be understood to not be limited in its application to the specific experimentation, results, and laboratory procedures disclosed herein. Rather, the Example is simply provided as one of various embodiments and are meant to be exemplary, not exhaustive.

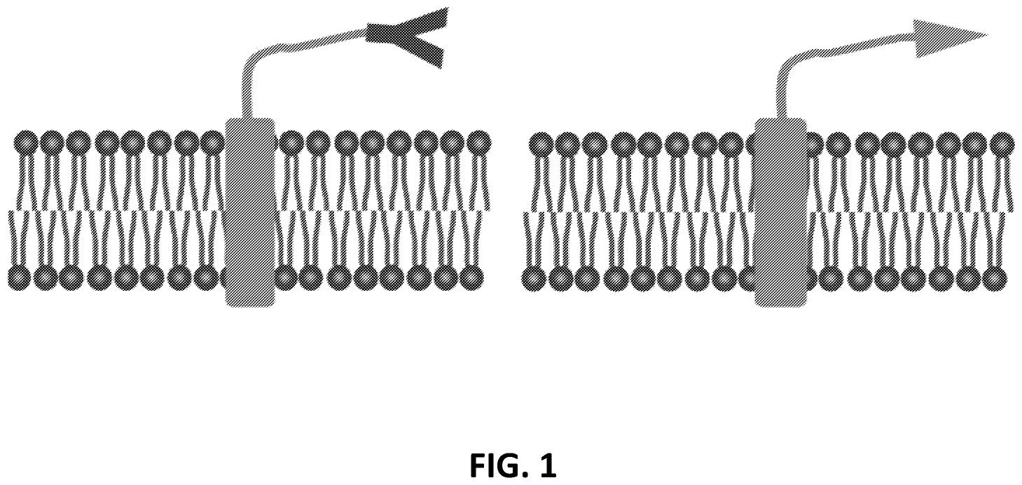

The present disclosure is based on the concepts that liposomes offer an environmentally friendly alternative to latex beads (microplastic) and that liposomes also allow integration of compounds that bind to or integrate into membranes. Binders that contain such domains can be attached to the liposomes. Thus, in the diagnostic reagent compositions of the present disclosure, liposomes are functionalized when such binders are applied that contain respective domains that bind to or integrate into membranes (FIG. 1). The binders are engineered through the combination of analyte-specific binding elements (such as, but not limited to, antigens, antibodies, ligands, etc.) with components or domains that bind to or integrate into membranes.

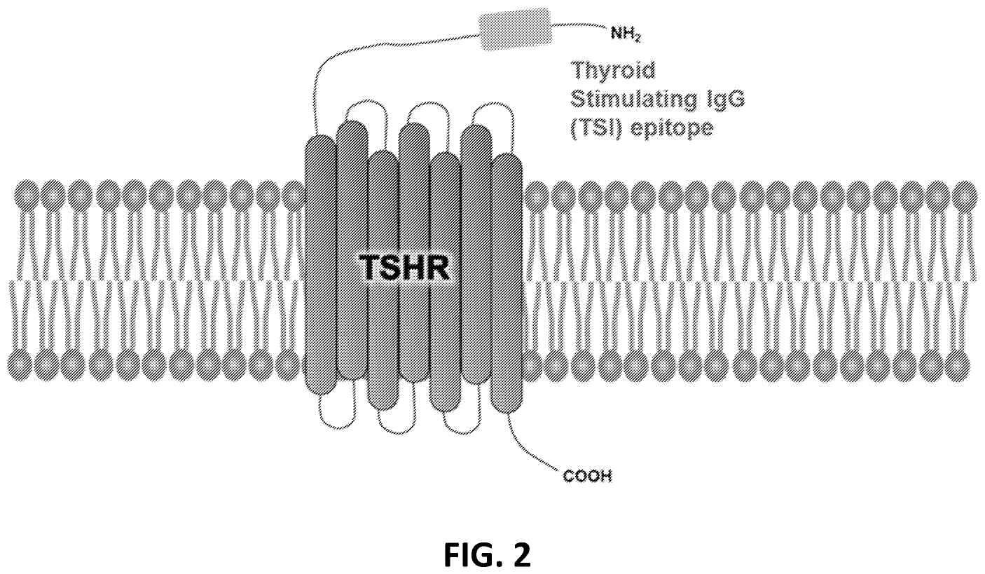

For example, the use of liposomes can allow for transmembrane domain-containing receptor proteins like Thyroid-Stimulating Hormone Receptor (TSHR) to naturally anchor in the lipid bilayer, exposing their extracellular domains for binding (FIG. 2) in a homogeneous immunoassay format. This arrangement mimics native membrane conditions for the receptor proteins. Alternatively, a conjugate can be produced that includes an extracellular, analyte-specific binding domain (i.e., an antigen, antibody, ligand, etc.) conjugated to a transmembrane domain.

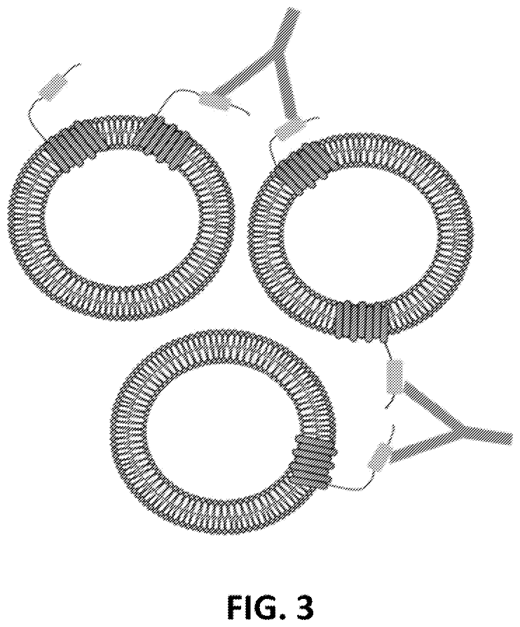

Liposomes are prepared in various sizes that encompass the sizes of NMLP's or latex beads typically used. TSHR has an extracellular thyroid stimulating immunoglobulin (TSI) binding epitope. When the transmembrane domain containing TSHR and liposomes are incubated together, TSHR-liposomes are formed. The functionalized vesicles (TSHR-liposomes) are then used in a homogeneous immuno-agglutination assay for detecting TSI (FIG. 3). Detectors can include (but are not limited to) a spectrophotometer, nephelometer, turbidimeter, Coulter counter, dynamic light scattering analyzer, flow cytometry-based systems using light scattering, and the like.

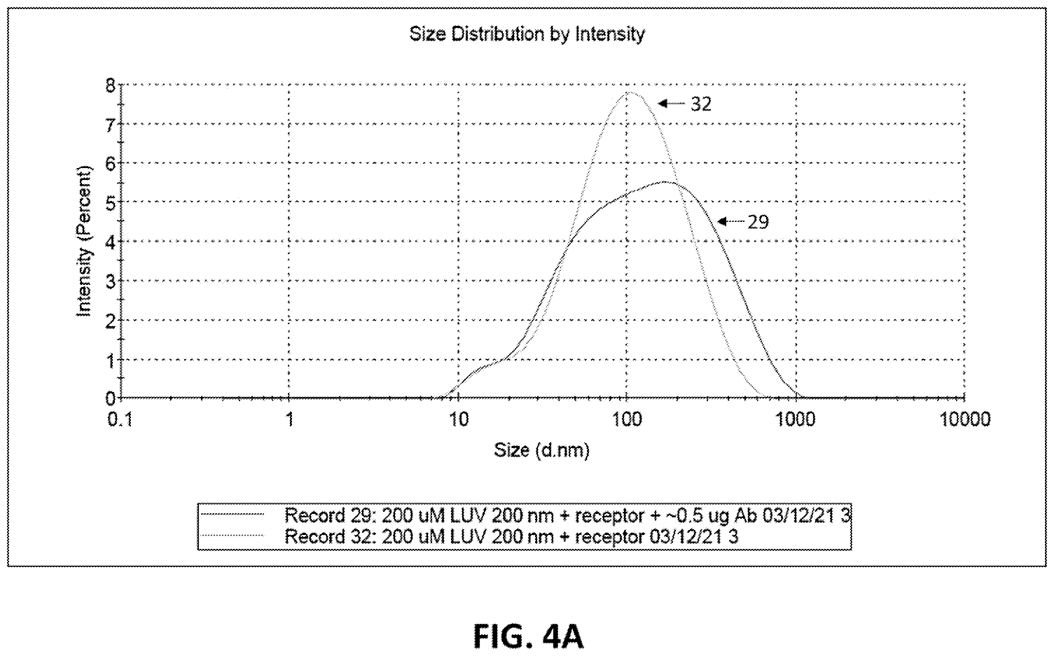

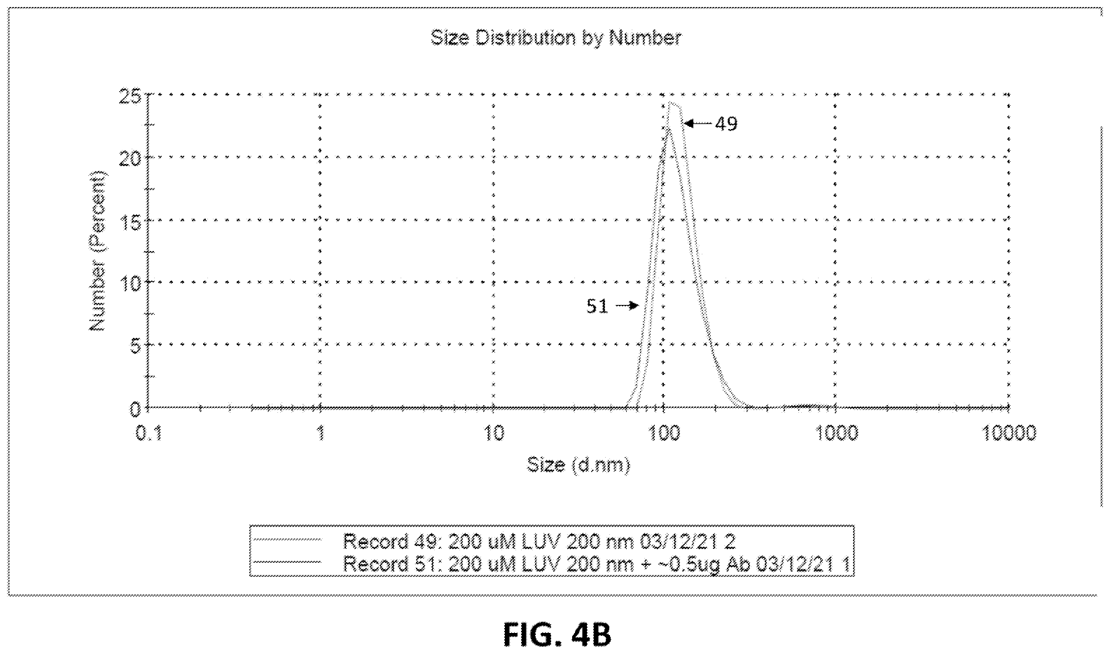

To demonstrate this concept, liposomes (100-200 nm) were prepared, incubated with TSHR, and tested using a commercial TSI (an anti-TSHR monoclonal antibody) on a dynamic light scattering analyzer (FIGS. 4A and 4B). FIG. 4A shows dynamic light scattering profiles of liposomes functionalized with TSHR (TSHR-liposomes) in the absence and presence of an anti-TSHR monoclonal antibody (TSI). The observed increase in size only after adding the antibody (TSI) demonstrates that an agglutination event occurred. Dynamic light scattering profiles of liposomes that are not functionalized with TSHR in the absence and presence of an anti-TSHR monoclonal antibody (TSI) show no increase in size after adding the antibody to the liposomes (FIG. 4B). Thus, there is no agglutination. This demonstrates that the agglutination event shown in FIG. 4A was specific and only occurred when functionalized TSHR-liposomes were used, thereby forming immune complexes with the TSI as represented in FIG. 3.

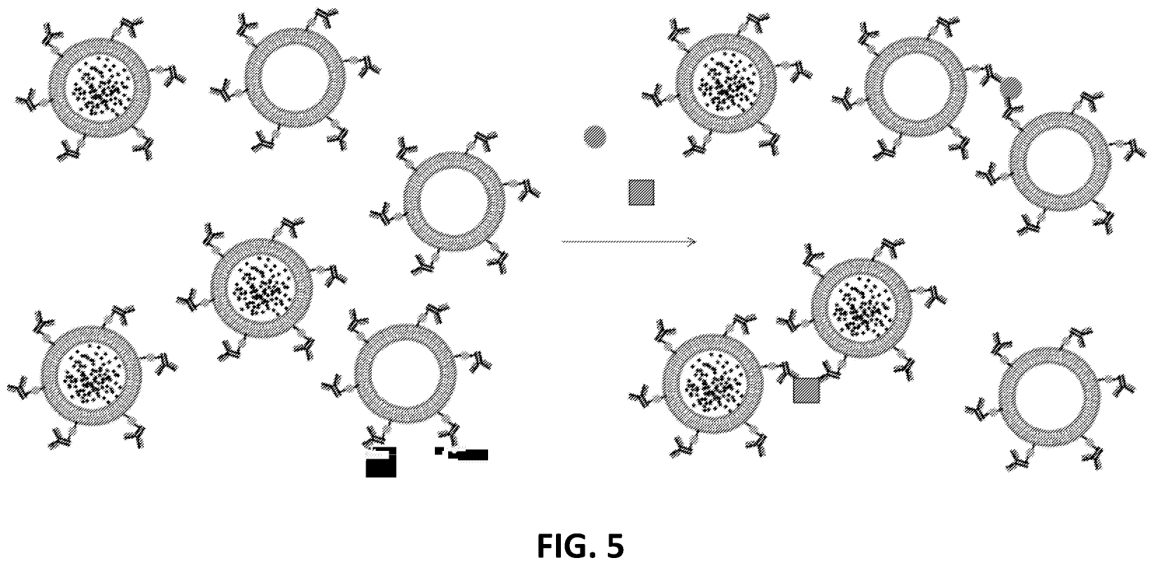

Furthermore, use of dye-encapsulating liposomes or dye-containing phospholipids enhances visibility and detection of complexes, especially when an assay involves a formation of visible clumps. These dyes-containing functionalized liposomes also allow for multiplexing, i.e., simultaneous detection of multiple analytes in a single reaction. As shown in FIG. 5, a first diagnostic reagent composition that has a first dye encapsulated therein and that binds to a first target square analyte (depicted as a square) can be provided in the same reaction with a second diagnostic reagent composition that binds to a second target analyte (depicted as a circle) and that is detected via another dye encapsulated therein or incorporated into the liposome. An example of a multiplexing analyzer can be a flow cytometry-based instrument (i.e., flow cytometers based on luminescent detection) or a lateral flow immunoassay device.

Functionalized liposomes like TSHR-liposomes or generally receptor-domain-containing binder liposomes described herein can replace the latex beads for homogeneous immunoassays.

The liposomes allow the transmembrane domain containing receptor proteins or binders like TSHR to naturally anchor in the lipid bilayer, exposing their extracellular binding domains for immunoassays. This mimics native membrane conditions for the receptor proteins and forgoes the need to use hazardous chemicals for attachment.

Use of dye-encapsulating liposomes or dye-containing phospholipids in the preparation of receptor-domain-containing binder liposomes can enhance visibility and detection as well as make multiplexing testing feasible.

### Non-Limiting Illustrative Embodiments

The following is a list of non-limiting illustrative embodiments disclosed herein:

Illustrative embodiment 1. A diagnostic immunoassay reagent composition for detection of a target analyte in a biological sample, comprising: a liposome having a lipid bilayer and an outer surface; at least one analyte-specific binding element that comprises a transmembrane domain and an extramembrane domain, wherein the extramembrane domain comprises a domain that specifically binds to the target analyte; and wherein the transmembrane domain of the at least one analyte-specific binding element is anchored in the lipid bilayer of the liposome so that at least a portion of the extramembrane domain of the analyte-specific binding element is exposed on the outer surface of the liposome for detection of the target analyte.

Illustrative embodiment 2. The diagnostic reagent composition of illustrative embodiment 1, wherein the at least one analyte-specific binding element is at least one receptor protein or portion or derivative thereof.

Illustrative embodiment 3. The diagnostic reagent composition of illustrative embodiment 2, wherein the at least one receptor protein or portion or derivative thereof is selected from the group consisting of at least a portion of thyroid-stimulating hormone receptor (TSHR), a ligand-gated ion channel receptor, an enzyme-linked or kinase-linked receptor, a cell-matrix adhesion receptor, a cell adhesion molecule, an adrenergic receptor, an lgG Fc receptor, a glucocorticoid receptor, a G protein-coupled receptor, and combinations thereof.

Illustrative embodiment 4. The diagnostic reagent composition of any one of illustrative embodiments 1-3, wherein the at least one analyte-specific binding element comprises a conjugate in which the domain that specifically binds to the target analyte is attached to the transmembrane domain.

Illustrative embodiment 5. The diagnostic reagent composition of illustrative embodiment 4, wherein the domain that specifically binds to the target analyte is a ligand or portion thereof.

Illustrative embodiment 6. The diagnostic reagent composition of illustrative embodiment 4, wherein the domain that specifically binds to the target analyte is an antibody or portion thereof.

Illustrative embodiment 7. The diagnostic reagent composition of any one of illustrative embodiments 1-6, wherein the liposome has a size in a range of from about 50 nm to about 1000 nm.

Illustrative embodiment 8. The diagnostic reagent composition of illustrative embodiment 7, wherein the liposome has a size in a range of from about 100 nm to about 200 nm.

Illustrative embodiment 9. The diagnostic reagent composition of any one of illustrative embodiments 1-8, wherein the liposome has at least one dye encapsulated therein.

Illustrative embodiment 10. The diagnostic reagent composition of any one of illustrative embodiments 1-9, wherein the liposome comprises at least one lipid selected from the group consisting of a phospholipid, sphingolipid, glycerophospholipid, and combinations thereof.

Illustrative embodiment 11. The diagnostic reagent composition of illustrative embodiment 10, wherein the liposome further comprises at least one of a sterol or a sterol derivative.

Illustrative embodiment 12. The diagnostic reagent composition of any one of illustrative embodiments 1-11, wherein the liposome comprises at least one dye-containing phospholipid.

Illustrative embodiment 13. The diagnostic reagent composition of any one of illustrative embodiments 1-12, wherein the liposome comprises at least one biotinylated phospholipid.

Illustrative embodiment 14. A kit, comprising: at least one diagnostic reagent composition of any one of illustrative embodiments 1-13.

Illustrative embodiment 15. The kit of illustrative embodiment 14, further comprising at least one additional reagent for use in the diagnostic immunoassay.

Illustrative embodiment 16. The kit of illustrative embodiment 14 or 15, further comprising at least two diagnostic reagent compositions, and wherein the kit is for use in a multiplexed assay.

Illustrative embodiment 17. A microfluidics device, comprising: (i) an inlet channel through which a sample is applied; and (ii) at least one compartment capable of being in fluidic communication with the inlet channel, wherein the at least one compartment comprises at least one diagnostic reagent composition of any one of illustrative embodiments 1-13.

Illustrative embodiment 18. The microfluidics device of illustrative embodiment 17, further defined as a microfluidics device for performing a multiplexed assay, and wherein (ii) comprises at least two diagnostic reagent compositions.

Illustrative embodiment 19. A method of producing a diagnostic reagent composition for detection of a target analyte in a biological sample, the method comprising the steps of: (1) combining at least one lipid with at least one analyte-specific binding element to form a mixture, wherein the at least one analyte-specific binding element comprises a transmembrane domain and an extramembrane domain, and wherein the extramembrane domain comprises a domain that specifically binds to the target analyte; and (2) incubating the mixture under conditions that result in the formation of at least one liposome having the at least one analyte-specific binding element anchored in a lipid bilayer of the liposome via the transmembrane domain of the at least one analyte-specific binding element, and wherein the extramembrane domain of the at least one analyte-specific binding element comprises a domain that extends from an outer surface of the liposome for specifically binding to the target analyte.

Illustrative embodiment 20. The method of illustrative embodiment 19, wherein the at least one analyte-specific binding element is selected from the group consisting of at least one receptor protein or portion thereof and a conjugate in which the domain that specifically binds to the target analyte is attached to a transmembrane domain.

Illustrative embodiment 21. The method of illustrative embodiment 20, wherein: the at least one analyte-specific binding element is at least one receptor protein or portion thereof selected from the group consisting of at least a portion of thyroid-stimulating hormone receptor (TSHR), a ligand-gated ion channel receptor, an enzyme-linked or kinase-linked receptor, a cell-matrix adhesion receptor, a cell adhesion molecule, an adrenergic receptor, an lgG Fc receptor, a glucocorticoid receptor, a G protein-coupled receptor, and combinations thereof; the at least one analyte-specific binding element is a conjugate, and the domain that specifically binds to the target analyte is a ligand or portion thereof; or the at least one analyte-specific binding element is a conjugate, and the domain that specifically binds to the target analyte is an antibody or portion thereof.

Illustrative embodiment 22. The method of any one of illustrative embodiments 19-21, wherein the liposome has a size in a range of from about 50 nm to about 1000 nm.

Illustrative embodiment 23. The method of illustrative embodiment 22, wherein the liposome has a size in a range of from about 100 nm to about 200 nm.

Illustrative embodiment 24. The method of any one of illustrative embodiments 19-23, wherein the at least one lipid of step (1) is selected from the group consisting of a phospholipid, sphingolipid, glycerophospholipid, and combinations thereof.

Illustrative embodiment 25. The method of illustrative embodiment 24, wherein the at least one lipid further comprises at least one of a sterol or a sterol derivative.

Illustrative embodiment 26. The method of any one of illustrative embodiments 19-25, wherein in step (1), at least one dye is added to the mixture such that the at least one liposome formed in step (2) has at least one dye encapsulated therein.

Illustrative embodiment 27. The method of any one of illustrative embodiments 19-26, wherein the at least one lipid of step (1) comprises at least one dye-containing phospholipid and/or at least one biotinylated phospholipid.

Illustrative embodiment 28. A method of producing a diagnostic reagent composition for detection of a target analyte in a biological sample, the method comprising the steps of: (1) combining at least one liposome with at least one analyte-specific binding element to form a mixture, wherein the at least one liposome has a lipid bilayer and an outer surface, wherein the at least one analyte-specific binding element comprises a transmembrane domain and an extramembrane domain, and wherein the extramembrane domain comprises a domain that specifically binds to the target analyte; and (2) incubating the mixture under conditions that result in the anchoring of the at least one analyte-specific binding element in the lipid bilayer of the at least one liposome via the transmembrane domain of the at least one analyte-specific binding element, and wherein the extramembrane domain of the at least one analyte-specific binding element comprises a domain that extends from the outer surface of the liposome for specifically binding to the target analyte.

Illustrative embodiment 29. The method of illustrative embodiment 28, wherein the at least one analyte-specific binding element is selected from the group consisting of at least one receptor protein or portion thereof and a conjugate in which the domain that specifically binds to the target analyte is attached to a transmembrane domain.

Illustrative embodiment 30. The method of illustrative embodiment 29, wherein: the at least one analyte-specific binding element is at least one receptor protein or portion thereof selected from the group consisting of at least a portion of thyroid-stimulating hormone receptor (TSHR), a ligand-gated ion channel receptor, an enzyme-linked or kinase-linked receptor, a cell-matrix adhesion receptor, a cell adhesion molecule, an adrenergic receptor, an lgG Fc receptor, a glucocorticoid receptor, a G protein-coupled receptor, and combinations thereof; the at least one analyte-specific binding element is a conjugate, and the domain that specifically binds to the target analyte is a ligand or portion thereof; or the at least one analyte-specific binding element is a conjugate, and the domain that specifically binds to the target analyte is an antibody or portion thereof.

Illustrative embodiment 31. The method of any one of illustrative embodiments 27-30, wherein the liposome has a size in a range of from about 50 nm to about 1000 nm.

Illustrative embodiment 32. The method of illustrative embodiment 31, wherein the liposome has a size in a range of from about 100 nm to about 200 nm.

Illustrative embodiment 33. The method of any one of illustrative embodiments 27-32, wherein the liposome comprises at least one lipid selected from the group consisting of a phospholipid, sphingolipid, glycerophospholipid, and combinations thereof.

Illustrative embodiment 34. The method of illustrative embodiment 33, wherein the liposome further comprises at least one of a sterol or a sterol derivative.

Illustrative embodiment 35. The method of any one of illustrative embodiments 27-34, wherein the at least one liposome has at least one dye encapsulated therein.

Illustrative embodiment 36. The method of any one of illustrative embodiments 27-35, wherein the at least one liposome comprises at least one dye-containing phospholipid and/or at least one biotinylated phospholipid.

Illustrative embodiment 37. A method of determining the presence and/or concentration of a target analyte in a biological sample, the method comprising the steps of: combining the biological sample with at least one diagnostic reagent composition of any one of illustrative embodiments 1-13 under conditions that allow for binding of the at least one analyte-specific binding element to target analyte present in the sample to form a complex; and determining the presence and/or concentration of the target analyte based on any complex formed.

Illustrative embodiment 38. The method of illustrative embodiment 37, wherein the method comprises a homogeneous assay format.

Illustrative embodiment 39. The method of illustrative embodiment 38, wherein the assay format comprises an agglutination assay.

Illustrative embodiment 40. The method of any one of illustrative embodiments 37-39, wherein the method comprises a heterogeneous assay format.

Illustrative embodiment 41. The method of illustrative embodiment 40, wherein the assay format comprises a sandwich assay.

Illustrative embodiment 42. A method of determining the presence and/or concentration of at least two target analytes in a biological sample, the method comprising the steps of: combining the biological sample with a first diagnostic reagent composition of any one of illustrative embodiments 1-13 and a second diagnostic reagent composition of any of illustrative embodiments 1-13 under conditions that allow for binding of the analyte-specific binding element of the first diagnostic reagent composition to first target analyte present in the sample to form a first complex and that allow for binding of the analyte-specific binding element of the second diagnostic reagent composition to the second target analyte present in the sample to form a second complex; determining the presence and/or concentration of the first target analyte based on any first complex formed; and determining the presence and/or concentration of the second target analyte based on any second complex formed.

Thus, in accordance with the present disclosure, there have been provided compositions, kits, and devices, as well as methods of producing and using same, which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

1. A diagnostic immunoassay reagent composition for detection of a target analyte in a biological sample, comprising:
a liposome having a lipid bilayer and an outer surface;
at least one analyte-specific binding element that comprises a transmembrane domain and an extramembrane domain, wherein the extramembrane domain comprises a domain that specifically binds to the target analyte; and
wherein the transmembrane domain of the at least one analyte-specific binding element is anchored in the lipid bilayer of the liposome so that at least a portion of the extramembrane domain of the analyte-specific binding element is exposed on the outer surface of the liposome for detection of the target analyte.

2. The diagnostic reagent composition of claim 1, wherein the at least one analyte-specific binding element is at least one receptor protein or portion or derivative thereof.

3. The diagnostic reagent composition of claim 2, wherein the at least one receptor protein or portion or derivative thereof is selected from the group consisting of at least a portion of thyroid-stimulating hormone receptor (TSHR), a ligand-gated ion channel receptor, an enzyme-linked or kinase-linked receptor, a cell-matrix adhesion receptor, a cell adhesion molecule, an adrenergic receptor, an lgG Fc receptor, a glucocorticoid receptor, a G protein-coupled receptor, and combinations thereof.

4. The diagnostic reagent composition of claim 1, wherein the at least one analyte-specific binding element comprises a conjugate in which the domain that specifically binds to the target analyte is attached to the transmembrane domain.

5. The diagnostic reagent composition of claim 4, wherein the domain that specifically binds to the target analyte is a ligand or portion thereof.

6. The diagnostic reagent composition of claim 4, wherein the domain that specifically binds to the target analyte is an antibody or portion thereof.

7. The diagnostic reagent composition of claim 1, wherein the liposome has a size in a range of from about 50 nm to about 1000 nm.

8. The diagnostic reagent composition of claim 7, wherein the liposome has a size in a range of from about 100 nm to about 200 nm.

9. The diagnostic reagent composition of claim 1, wherein the liposome has at least one dye encapsulated therein.

10. The diagnostic reagent composition of claim 1, wherein the liposome comprises at least one lipid selected from the group consisting of a phospholipid, sphingolipid, glycerophospholipid, and combinations thereof.

11. The diagnostic reagent composition of claim 10, wherein the liposome further comprises at least one of a sterol or a sterol derivative.

12. The diagnostic reagent composition of claim 1, wherein the liposome comprises at least one dye-containing phospholipid.

13. The diagnostic reagent composition of claim 1, wherein the liposome comprises at least one biotinylated phospholipid.

14. A kit, comprising:
the diagnostic reagent composition of claim 1.

15. The kit of claim 14, further comprising at least one additional reagent for use in the diagnostic immunoassay.

16. The kit of claim 14, further comprising at least two diagnostic reagent compositions, and wherein the kit is for use in a multiplexed assay.

17. A microfluidics device, comprising:
(i) an inlet channel through which a sample is applied; and
(ii) at least one compartment capable of being in fluidic communication with the inlet channel, wherein the at least one compartment comprises the diagnostic reagent composition of claim 1.

18. The microfluidics device of claim 17, further defined as a microfluidics device for performing a multiplexed assay, and wherein (ii) comprises at least two diagnostic reagent compositions.

19. A method of producing a diagnostic reagent composition for detection of a target analyte in a biological sample, the method comprising the steps of:
(1) combining at least one lipid with at least one analyte-specific binding element to form a mixture, wherein the at least one analyte-specific binding element comprises a transmembrane domain and an extramembrane domain, and wherein the extramembrane domain comprises a domain that specifically binds to the target analyte; and
(2) incubating the mixture under conditions that result in the formation of at least one liposome having the at least one analyte-specific binding element anchored in a lipid bilayer of the liposome via the transmembrane domain of the at least one analyte-specific binding element, and wherein the extramembrane domain of the at least one analyte-specific binding element comprises a domain that extends from an outer surface of the liposome for specifically binding to the target analyte.

20. The method of claim 19, wherein the at least one analyte-specific binding element is selected from the group consisting of at least one receptor protein or portion thereof and a conjugate in which the domain that specifically binds to the target analyte is attached to a transmembrane domain.

21.-41. (canceled)

42. A method of determining the presence and/or concentration of at least two target analytes in a biological sample, the method comprising the steps of:
combining the biological sample with a first diagnostic reagent composition and a second diagnostic reagent composition under conditions that allow for binding of the analyte-specific binding element of the first diagnostic reagent composition to first target analyte present in the sample to form a first complex and that allow for binding of the analyte-specific binding element of the second diagnostic reagent composition to the second target analyte present in the sample to form a second complex;
determining the presence and/or concentration of the first target analyte based on any first complex formed; and
determining the presence and/or concentration of the second target analyte based on any second complex formed.
